(19) United States
(12) Patent Application Publication
(10) Pub. No.: US 2007/0167687 A1
(43) Pub. Date: Jul. 19, 2007

(54) Systems and methods for disease management

(76) Inventors: Arthur A. Bertolero, Danville, CA (US); Kent Richards, San Clemente, CA (US); Sing-Fatt Chin, Pleasanton, CA (US); Lon Annest, Tacoma, WA (US); Rob O'Reilly, Lafayette, CA (US)

Correspondence Address: GSS LAW GROUP, Suite 317, 3900 Newpark Mall Road, Newark, CA, 94560, US

(21) Appl. No.: 11/444,136

(22) Filed: May 30, 2006

Related U.S. Application Data
(60) Provisional application No. 60/685,753, filed on May 27, 2005.
(60) Provisional application No. 60/689,012, filed on Jun. 9, 2005.

(51) Int. Cl. A61B 5/00; G06Q 10/00; A61B 17/08

(52) U.S. Cl. 600/300; 606/153; 128/920; 705/2

(57) ABSTRACT

A web-based, interactive diagnosis, referral and reference system is used by physicians, patients, and others in the health care field to serve as a diagnostic, evaluation, and utilization tool/guide. The user may enter answer a series of guided questions to reach a diagnosis, obtain a physician referral and/or review literature and other reference materials on conditions. A method and device for left ventricular reconstruction is also disclosed. The device includes a curved needle or introducer which is inserted into one ventricle, through the septum and into the other ventricle. An anchor is then used to fold and hold the wall of the heart inward, thereby reducing the volume of the ventricle.

### CROSS REFERENCE TO OTHER APPLICATIONS

This application claims the benefit of U.S. provisional application Ser. No. 60/685,753 filed May 27, 2005 and of 60/689,012 filed Jun. 9, 2005. The disclosures of these applications are hereby incorporated by reference in their entirety.

The disclosures of the following applications are incorporated by reference in their entirety:

U.S. Ser. No. 08/766,384 filed Dec. 6, 1996

U.S. Ser. No. 09/145,016 filed Sep. 1, 1998

U.S. Ser. No. 09/171,064 filed Oct. 9, 1998

U.S. Ser. No. 09/171,206 filed Oct. 9, 1998

U.S. Ser. No. 09/672,110 filed Aug. 10, 1999

U.S. Ser. No. 10/371,756 filed Feb. 21, 2003

U.S. Ser. No. 10/996,876 filed Nov. 23, 2004

U.S. Ser. No. 11/133,142 filed May 19, 2005

U.S. Ser. No. 60/014,922 filed Apr. 10, 1996

PCT/US97/06112 filed Apr. 10, 1997

PCT/US97/06533 filed Apr. 10, 1997

### FIELD OF THE INVENTION

This invention relates generally to the field of disease management. More particularly, it relates to an artificial intelligence disease management system for use by hospitals, allied health personnel, physicians, insurance companies, health maintenance organizations (HMO's), patients, and others in the health care field to serve as a diagnostic, evaluation, and utilization tool/guide for health care provided to individuals in certain disease states who meet specific diagnostic or financial criteria or anyone from an individual to health care providers. And more particularly, for patient self-diagnosis and self-referral, such as for heart related diseases and conditions.

### BACKGROUND OF THE INVENTION

Due to the increasing complexity and cost of providing health/disease care, there is an ever increasing emphasis on managing the health care process. The process extends from an individual presenting a health concern to a health care provider and continues through diagnosis, therapeutic selection, resource selection, treatment options, and tracking or follow-up. This process could be extended further to include proactively identifying and preventing health concerns and planning for anticipated resource needs at one end of the process, and daily nursing management and disability management at the other end of the process.

Previous efforts to manage disease included manual-historical systems where individual files recording actual treatment provided were manually reviewed to collect statistics on general categories of care or to review the appropriateness of in a given case. Such methods are labor-intensive and inefficient, and prone to be inaccurate. Efforts have been made to standardize data collection forms, descriptions of conditions, descriptions of treatment, and treatments in order to more efficiently collect and evaluate health care data. Other efforts have been made to automate the analysis of historical health care data for persons with particular disease conditions. These efforts focus mainly on collecting financial data and serve accounting and administrative functions.

At least one known automated prior art health care management system addresses therapeutic selection by starting with a selected treatment and, based on patient information provided by the user, evaluating whether or not that treatment is appropriate. See “Designing a guide-line utilization management program”, Benefits Quarterly, 1991 4th Quarter; 7(4).42-47. Such systems do not develop a treatment based on various data describing an individual's health condition; the user or patient must first select a predefined treatment. Also, these systems do not have the flexibility to modify or add treatments based on individual's changing health condition. Further, these systems do not have an integral component whereby explanatory information is elicited from the health care provider or reviewer to facilitate analysis of the difference between actual or proposed treatment and developed treatment.

It would be a decided improvement over the prior art to have an artificial intelligence disease management system that could be used by various health care participants, including physicians, nurses, health care administrators, payor administrators, employers, evaluators, and patients at multiple stages of the health care process. It would be a further improvement for such a system to collect information on individuals having a health concern, to guide the user to a system-selected treatment based on the information collected, and to compare an actual or proposed treatment with the system-selected treatment.

It would be a further improvement over the prior art for such a system to permit continuous updating and modification of the experience base, using the information input into the process for each case. For example, the information on actual treatment provided can be used to reassess the decision path for system-selected treatments.

A system implementing the above process should have several elements. It should provide the guidance for physicians and nurses to perform the most appropriate assessment on patients. It should be cost-effective, i.e. lead to reducing the total cost of health care. It should be usable in real-time, i.e. the information input into the system should be immediately processed and available for further use. It should be flexible enough to adapt to changes in and evolution of health care professional knowledge and health care treatment methods, particularly in heart failure and atrial fibrillation. This system will enable patients to evaluate which treatment options they are interested in discussing with their health care provider based on factors such as risks and benefits, invasiveness versus cure rates, etc.

Thus, there is a need for an artificial intelligence disease management data processing system to guide and standardize the patient's path of care in the disease management process, using a database of best practice health care records and health condition guidelines, that include providing diagnosis, advised treatment and utilization information.

As a practical example in this application, a few of the specifics in the field of congestive heart failure (CHF) are used to demonstrate how a system and methods of the present invention would be used. However, the concepts described may be used in many other medical fields without departing from the scope of the present invention.

An estimated 4.8 million Americans suffer from CHF. CHF and related complications claim the lives of nearly 300,000 patients per year. Currently there are approximately 50,000 patients on the heart transplant list and less than 2,000 will undergo a heart transplantation. Despite the availability of left ventricular assist devices (LVADs), only a small number of patients (approximately 1,200), have been identified by a cardiologist, qualified, and agreed to undergo a LVAD placement procedure.

Despite its potential as an alternative in many advanced CHF patients, only a small number of patients, approximately 4,000, will be identified by cardiologists and referred to surgeons for the Dor procedure for left ventricular reconstruction (LVR). Both the Dor procedure for LVR and the LVAD devices have been used in hundreds of patients. Since 1985, the Dor procedure has been used to help end stage CHF patients, where the ventricle is opened, a Sizer is used to resize the new ventricle and a HEMASHIELD or other such patch is used to patch the opening resulting in the ventricle pumping much more efficiently. Peer reviewed journal articles have been published informing cardiologists and cardiac surgeons of this life saving procedure; yet, the number of patients treated using these procedures has grown only modestly over the last few years. The NIH has funded a clinical trial to prove to medical professionals that LVADs and LVR procedures can save thousands of lives. However, patient enrollment in this trial has progressed so slowly it will take years for this trial to be completed. In contrast, the average pacemaker trial with 1,000 patients is usually completed in 6 months to 1 year, where cardiologists are selecting the patient and referring them within their own cardiology community.

This procedure is performed by open heart surgery, with its attendant expense and extended convalescence. Therefore, it would be beneficial to have a method and system that could temporarily unload a ventricle of a patient in order to facilitate one or more surgical procedures on the heart and its related blood vessels. It would also be advantageous to have a method and device that could be permanently implanted to close off a portion of the ventricle to improve heart function. It would be further advantageous to have methods and systems that could perform these functions while allowing cardiac contractions to continue.

It is clear that the treatment of cardiac disease is inefficient in the referral of patients from primary care to cardiology and on to surgery or from surgery back to cardiology.

Therefore, this invention will use evidence based medicine in the form of the invention's patient tracking database, and the invention's clinical decision protocols and methods of disease management to effectively refer patients for the best treatment as proven by the invention's disease management systems. (i.e., A CHF patient and their doctor will know the optimal medical therapy as of the date of diagnosis and whether the patient is a candidate for a pacemaker, LVR procedure or other therapy).

Another area in which the present invention could assist in disease management is post-surgical or post-catheter treatment and outpatient care. Again the following information is specific to the CHF field of medicine, but the concepts are applicable to other fields.

Heart failure is the leading cause of heart failure admissions for patients over the age of 65, with over 4,900,000 Americans afflicted. It is estimated that the national expenditures directed toward the care of heart failure patients exceeds $28 billion. The majority of the cost is related to hospitalizations, amounting to approximately $17 billion annually. The crisis management approach has historically been applied in the care of heart failure patients, wherein patients suffer exacerbation, are admitted for the “quick fix” and released from the hospital. As they return to the lifestyle within which they suffered the exacerbation, they spin into the cycle of symptom recurrence and repeat hospitalization. It is estimated that the 30 day readmission rate among hospitalized patients is 25%, and 50% within 6 months. Recurrent hospital admissions are believed to be caused in large part by lack of adherence to prescribed treatments, both diet and medications, on the part of the patient.

Due to prolonged lengths of hospital stay in this very ill population, and a high incidence of readmission within 30 days, reimbursement under the DRG structure is often inadequate to cover the cost of care. Hospital systems are actively seeking alternative strategies for delivering cost effective care. Because of the magnitude and severity of this disorder, it has been targeted by third party payers. Demonstration projects are currently underway exploring models of payment for quality of care and provider performance. The JCAHO standards include quality indicators directed at minimum care standards for the acute treatment of heart failure for hospitalized patients. As these trends continue, it is likely that similar demands will be made of those rendering outpatient and chronic care as well. While the current standards focus largely on medications prescribed and instruction given at hospital discharge, it is likely that the impending accountability for long term outcomes will require augmented systems approaches to assure ongoing compliance with the plan of care prescribed.

The American Heart Association and the American College of Cardiology have published evidence-based guidelines for the management of chronic heart failure. These guidelines provide the basis from which regulatory standards emerge, and should be used to guide development of quality indicators in the outpatient setting as well.

The JCAHO have identified the following performance measures for hospitalized patients:

Patient Education in the Following Areas.
Weight monitoring
Diet
Discharge medications
What to do if symptoms worsen
Activity level
Follow up appointment
Left Ventricular Assessment
Smoking cessation
ACE Inhibitor/ARB agents for patients with LV systolic dysfunction

Current candidate performance measures that are under consideration for future measurement sets include.
90-day readmission or ED visit rate after index HF admission
Percentage of patients monitoring body weight at least three times weekly
Drug dose and frequency specification
Number of HF patients screened for and given influenza vaccination
Number of HF patients screened for and given pneumonia vaccination

These performance measures further suggest the migration of the spotlight from the inpatient facilities to the outpatient, chronic care providers.

The American College of Cardiology, the American Heart Association, and the Physician Consortium for Performance Improvement have responded to the call for performance measures through publication of the Heart Failure Physician Performance Measurement Set. This document outlines standards of care that address laboratory and left ventricular function assessments, weight and blood pressure measurement, assessment of volume status, activity level, cardiac examination, medication prescription and patient education.

Published studies have consistently shown the value of multidisciplinary approaches to the problem of chronic heart failure management. While there is suggestion of improved survival among patients treated in these programs, the primary outcome of interest has been recurrent hospitalization. Additionally, adherence to evidence-based guidelines through these programs have shown demonstrable improvement.

An analysis by Dr. Turina of various clinical trials that of 93,000 patients screened for the BARI, RITA, GABI, and EAST Trials only 3.4% were entered into the trials. In the ARTS trial only 0.04% were entered into the trial. So these trials and the trial that are underway currently do not any kind of “real world” situation. It is the opinion of Dr. Turina as published in the European Journal of Cardio-Thoracic Surgery in 2004 that a true “all comers” trial will never happen.

Dr. Dawkins the author of the above mentioned paper agrees with Dr. Turina that surgeons and cardiologists will never agree to randomize to a true all comers trial.

Furthermore it is well accepted that most cardiology trial regarding the use of stents and pace makers are conducted at a select group of centers. The hospitals and doctors have experience, other treatment technology, personnel, skills, and other resources not found in the average hospital. Therefore even if these hospitals agreed to a true all comers clinical trial the results would not be able to be duplicated by the average hospitals and average doctors.

There are numerous diseases and conditions in which surgical procedures are performed on the heart. These include various cardiovascular, neurosurgical, pulmonary and other interventional procedures, including repair or replacement of aortic, mitral and other heart valves, repair of septal defects, congenital defect repairs, pulmonary thrombectomy, coronary artery bypass grafting, angioplasty, atherectomy, treatment of aneurysms during left ventricular reconstruction, electrophysiological mapping and ablation, and neurovascular procedures. Performance of these procedures may require general anesthesia and temporarily unloading the heart thereby reducing the blood flow to the lungs and arterial system. In such procedures, the heart must be isolated from the remainder of the venous system.

Using current techniques, isolation of the heart from the remainder of the venous system is accomplished by placing a mechanical cross-clamp externally on the Vena Cava (Inferior or superior or both) so as to prevent non-oxygenated blood from the arms, neck, head, and remainder of the body to reach to lungs, and therefore less oxygenated blood will reach the heart to allow the heart to stay decompressed, which is useful for some cardiac procedures, particularly offpump left ventricular reconstruction procedures. U.S. Pat. No. 6,913,600 to Valley et al. described a balloon catheter, which is used to isolate the heart from the remainder of the arterial system. In this system, the cardiac contractions are completely ceased.

### SUMMARY OF THE INVENTION

This inventions provides hospitals, doctors, payers (Medicare, Industry and Governments) and patients with an analysis tool that uses methods of disease management to determine treatment, weighting and long term follow up, with selected weighted clinical papers/presentations to determine the best practices for individual hospitals and doctors, thereby allowing treatment and analysis of anyone wanting to enter the system. The invention enables the hospital to offer a comprehensive inpatient/outpatient approach to treat congestive heart failure. This invention continuously updates results so patients obtain up to the minute best practices.

This invention provide an automated system that patients can self-assess and self-generate individualized medical information for a patient based on information provided by the patient, and guide the patients to proper channel for treatment options.

The present invention provides a disease management data processing system that is real-time appropriate, and cost effective to manage the health care process. The system can be used by hospitals, physicians, insurance companies, HMO's, patients, and others in the health care field to promote cost-effective and appropriate disease management.

This invention then provides doctors and patients with a tool to objectively weigh the risks and benefits of various treatment options or combinations, and to predict the real outcome based on that hospital's actual experience versus the success of a select group of patients, treated by a select group of doctors, and followed for a short time frame.

The system of the present invention focuses on organizational, financial and clinical elements. In one embodiment of the invention, a multi-disciplinary consulting team may be assigned to a hospital or clinic. A team leader would take charge of implementing the various tools selected, as well as routinely contacting the clinic for progress reports.

The present invention may include an artificial intelligence disease management system for use by hospitals, allied health personnel, physicians, insurance companies, health maintenance organizations (HMO's), patients, and others in the health care field to serve as a diagnostic, evaluation, and utilization tool/guide for health care provided to individuals in certain disease states. In particular, the system is suitable for patients with heart related diseases and conditions to self-evaluate and self-refer themselves to a physician appropriate for treatment of their condition.

The present invention also relates patients having these conditions undergoing procedures using devices and methods for temporarily unloading a ventricle of a patient in order to facilitate surgical procedures on the heart and its related blood vessels. These devices may also be permanently implanted to close off a portion of the ventricle to improve heart function.

Further the system may be used to isolating the heart of a patient from the remainder of the venous system. The present invention overcomes the limitations of the prior art by unloading the ventricle during offpump surgery, particularly left ventricle reconstruction procedure.

One aspect of the present invention comprises unloading ventricle during the offpump left ventricular reconstruction. The device is a catheter-based or surgical-based dual-balloon construction to occlude Superior Vena Cava (SVC) and/or Inferior Vena Cava (IVC) simultaneously or independently.

### BRIEF DESCRIPTION OF THE DRAWING

FIG. 1 is a flowchart of the disease management system of the present invention.

FIG. 2 is a flowchart of a patient driven system for selection of treatment.

FIG. 3 is a flowchart of a web driven patient self-assessment and referral system.

FIG. 4 is a flowchart of an outreach and follow-up system.

FIGS. 5A-H are flowcharts showing how the present invention would function for various situation.

FIGS. 6A-H are a flow chart of the assessment questionnaires.

FIG. 7 is a flow chart of a web-based, interactive diagnosis, referral and reference system.

FIGS. 8A-C show a method and device for left ventricular reconstruction using a left ventricular approach.

FIGS. 9A-C show an alternate procedure of FIGS. 8A-C.

FIGS. 10A-B show a method and device for left ventricular reconstruction using a right ventricular approach.

FIG. 11 shows an embodiment of the invention being used for unloading of the heart ventricle.

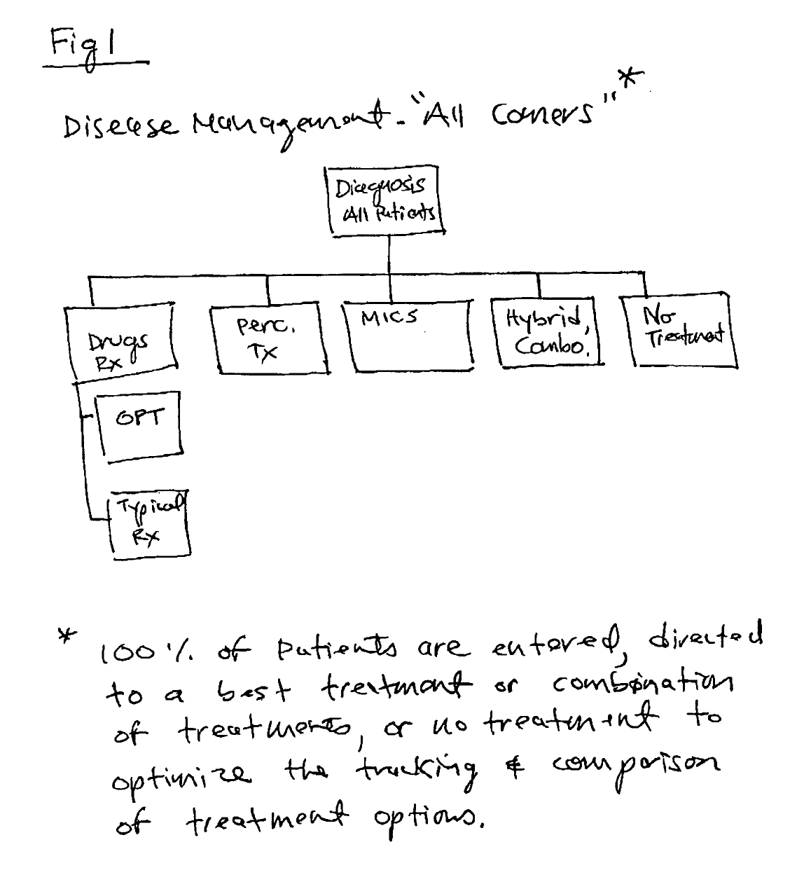

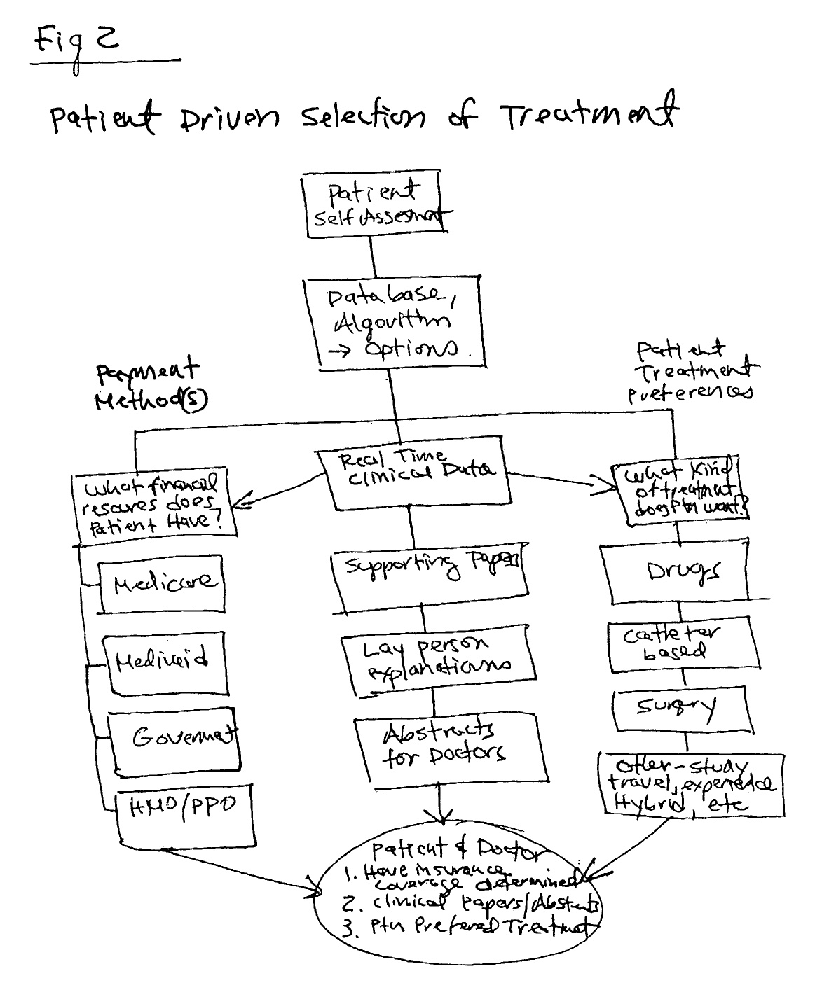

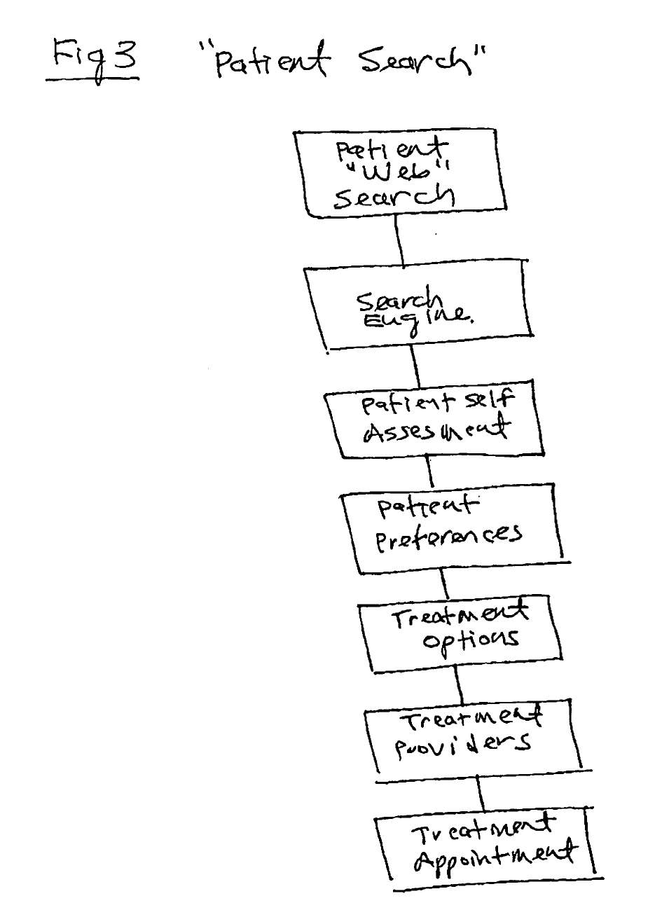

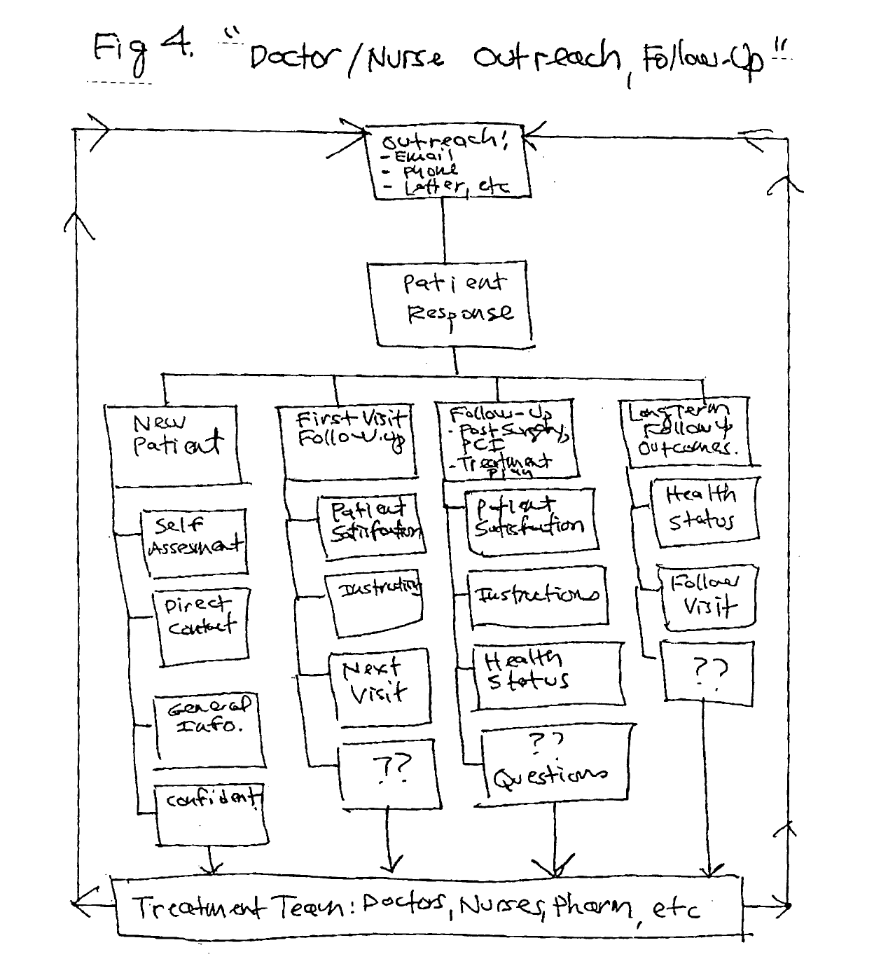

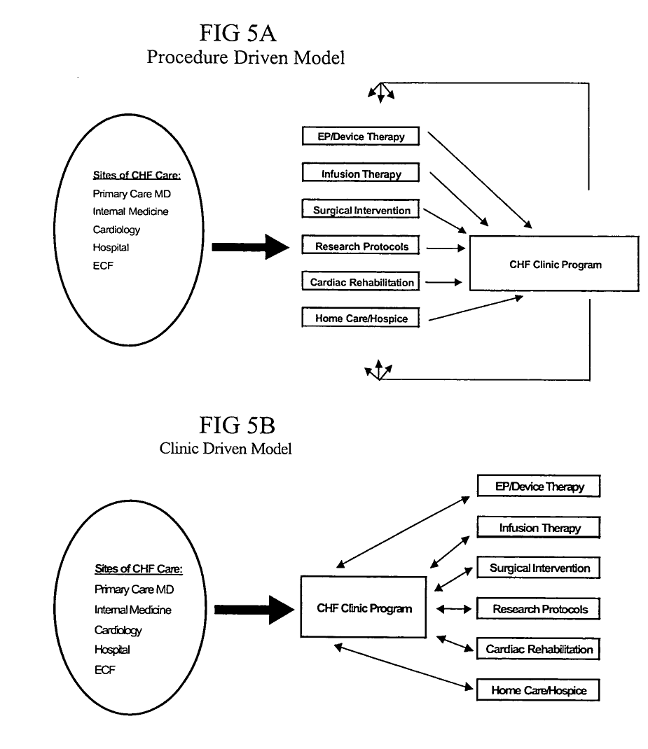

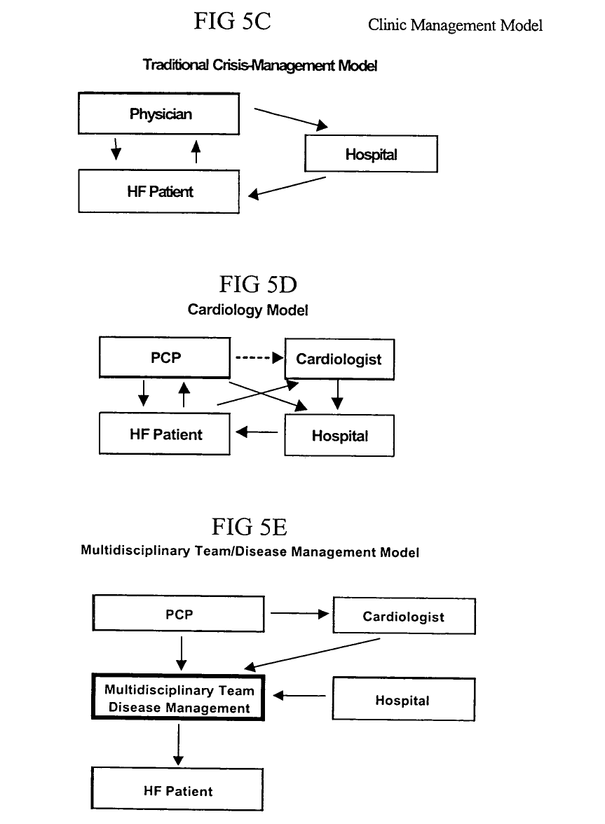

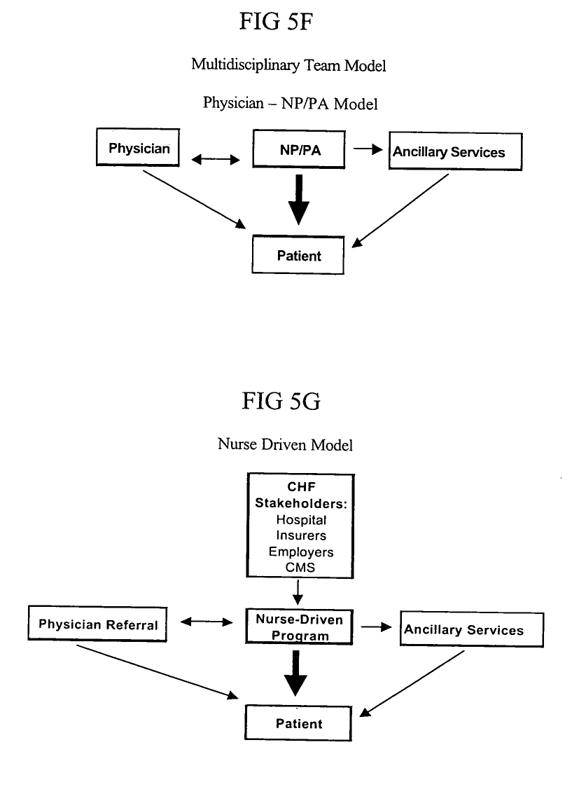

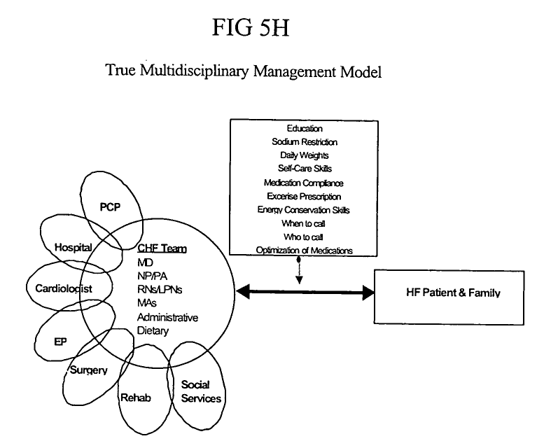

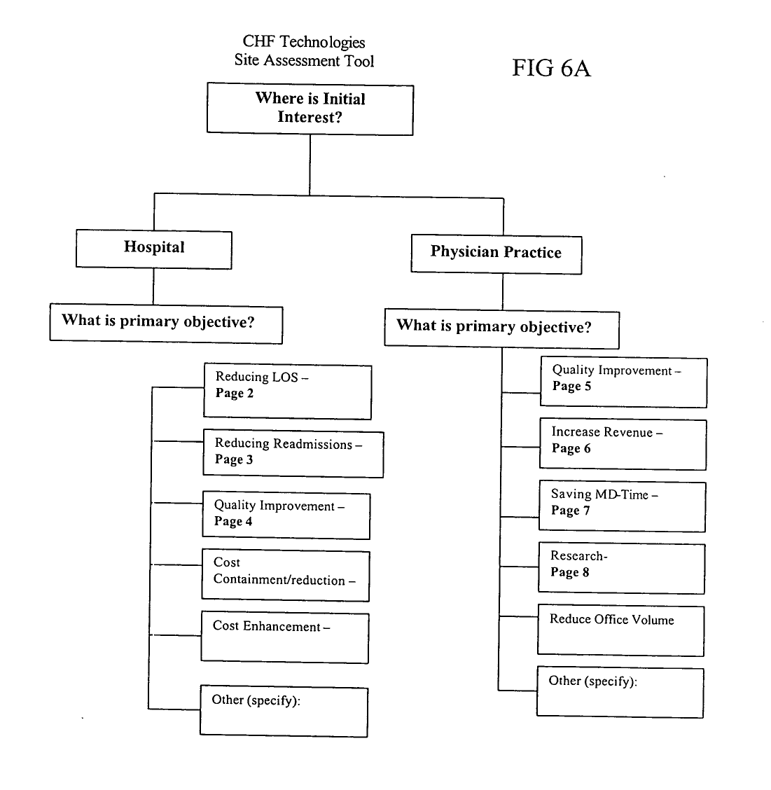

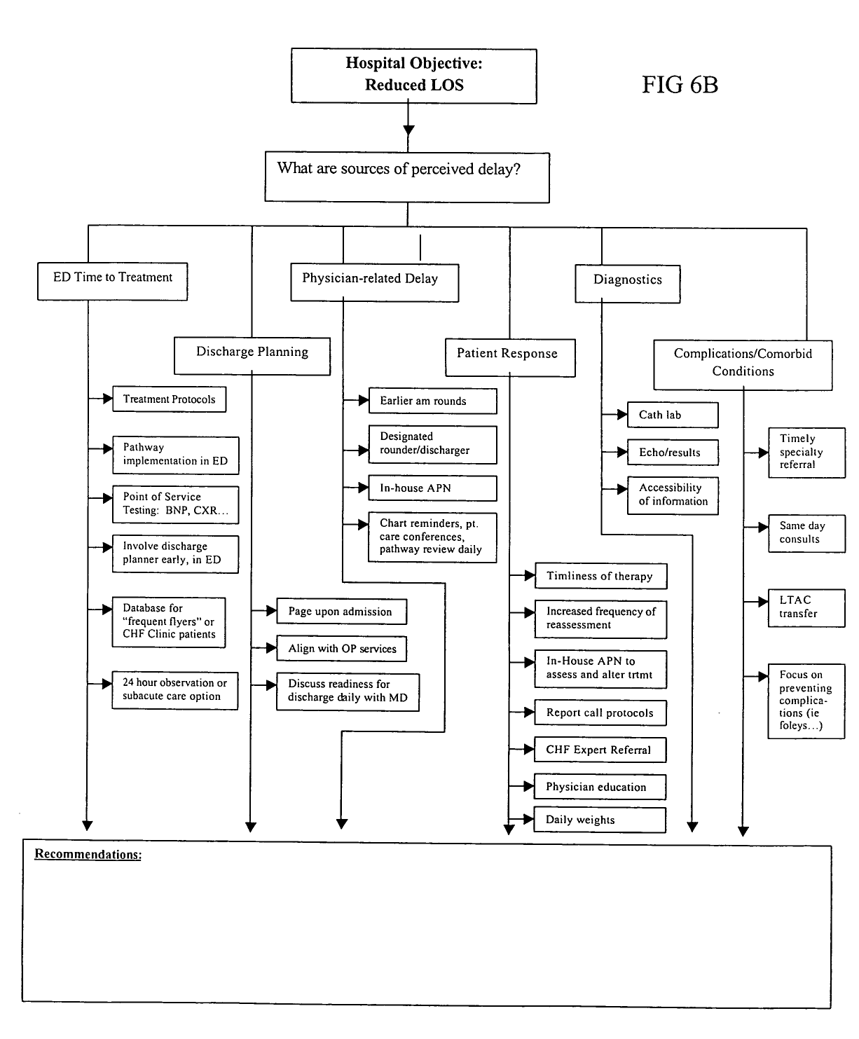

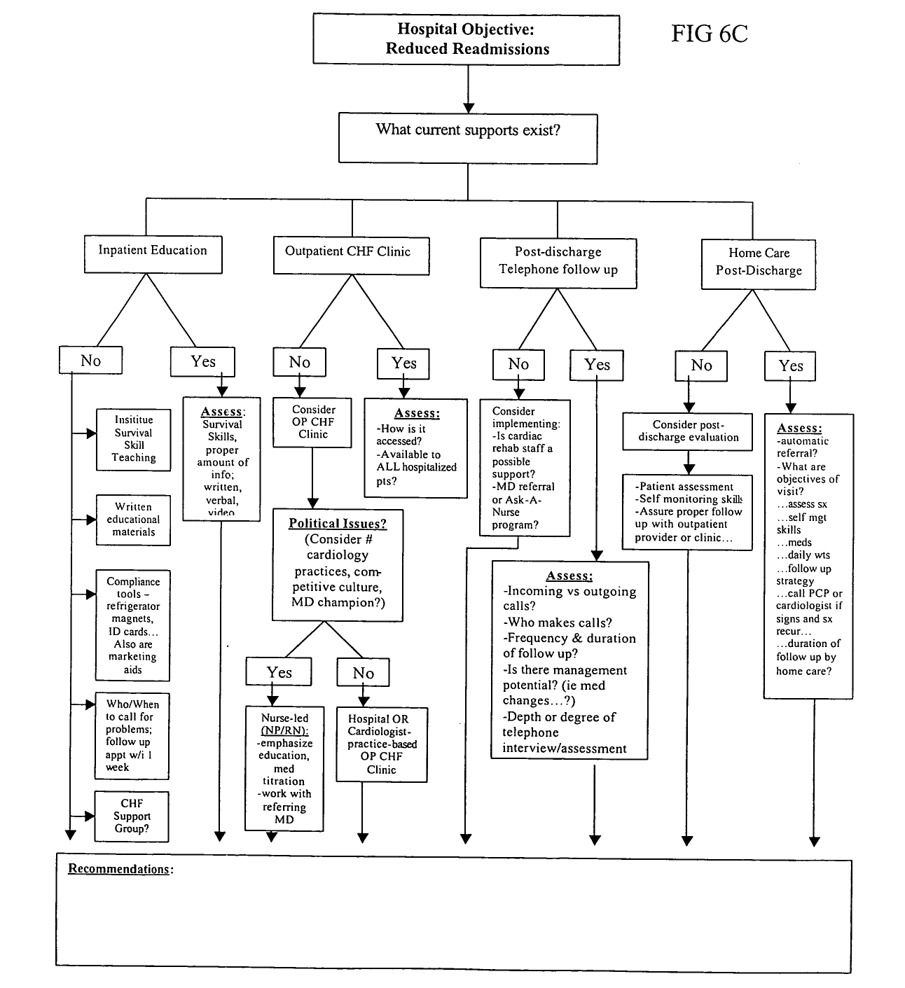

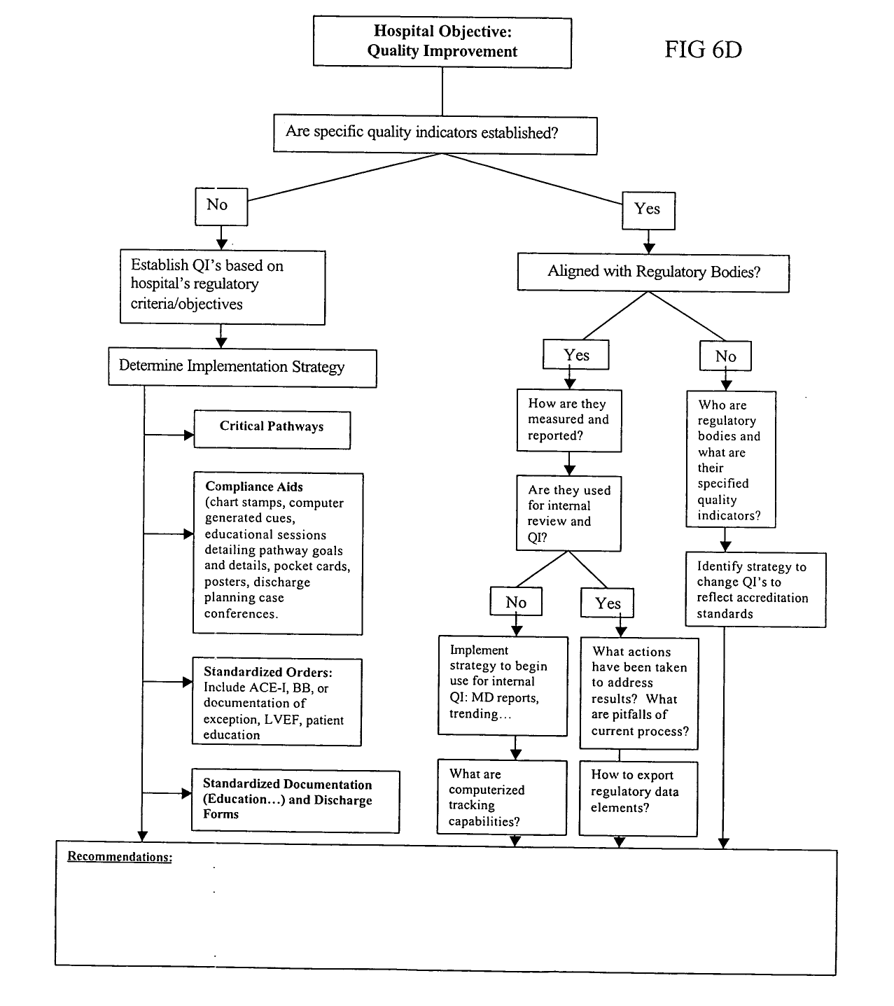

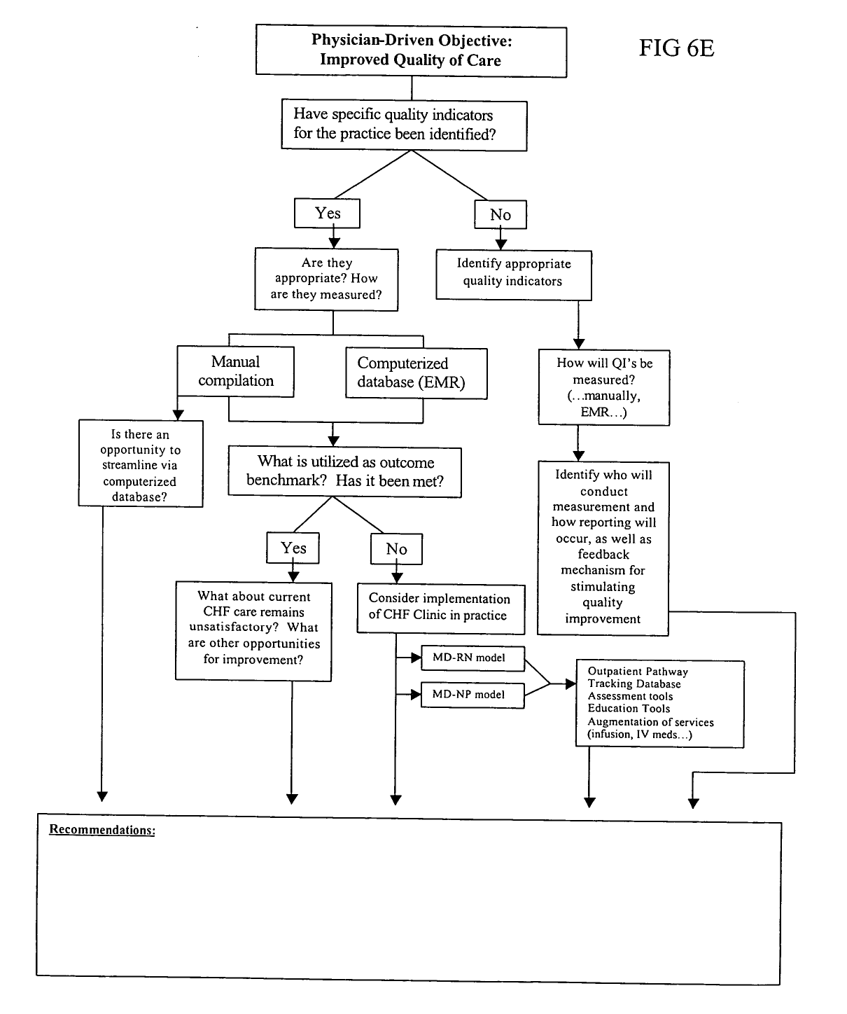

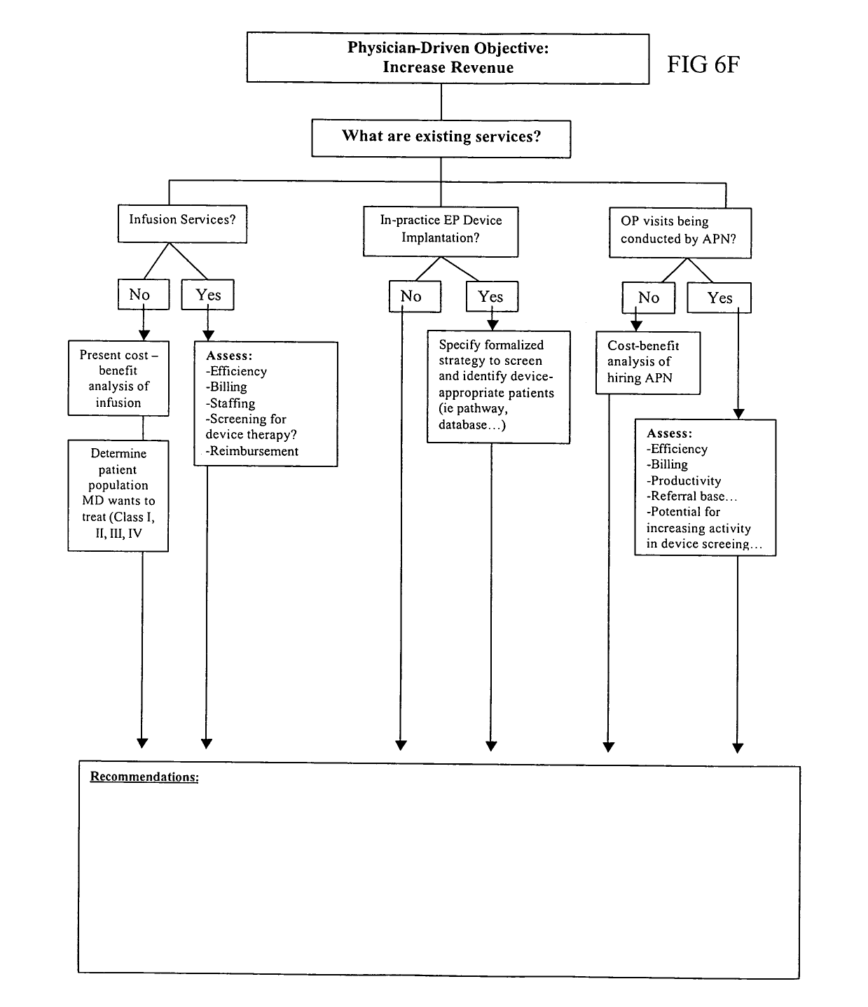

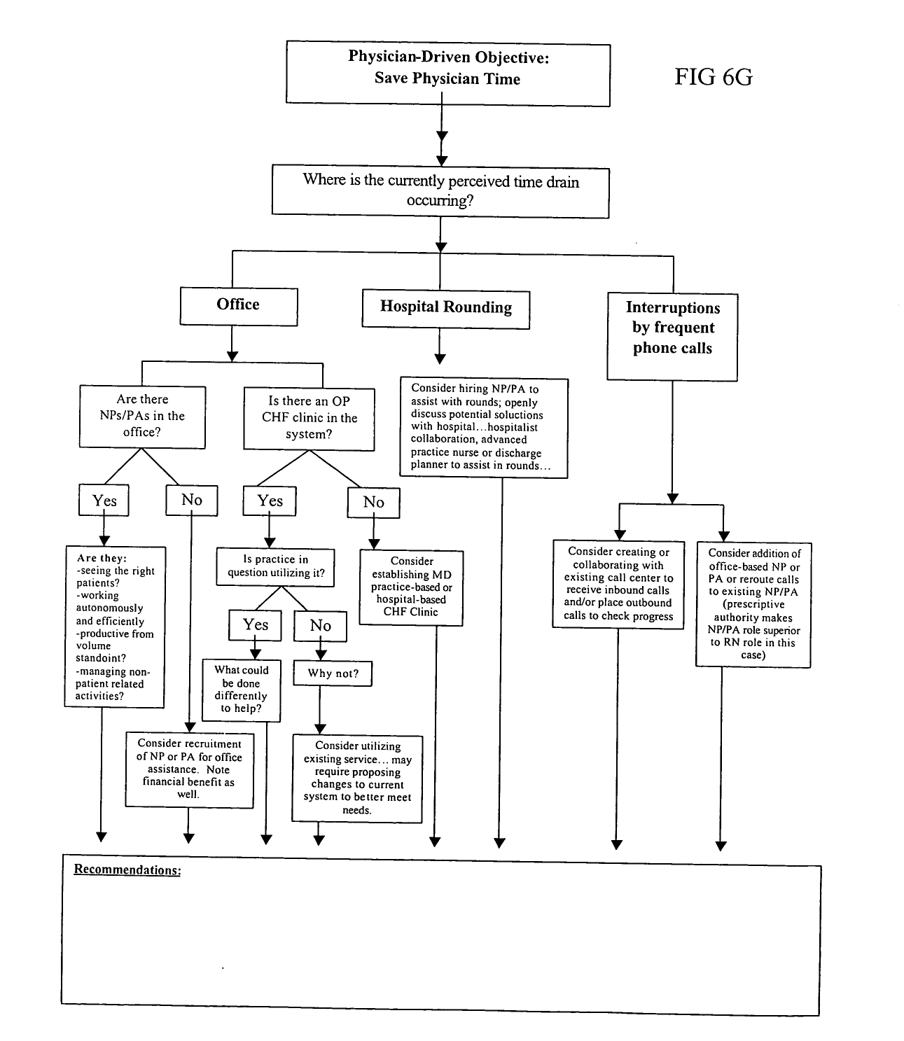

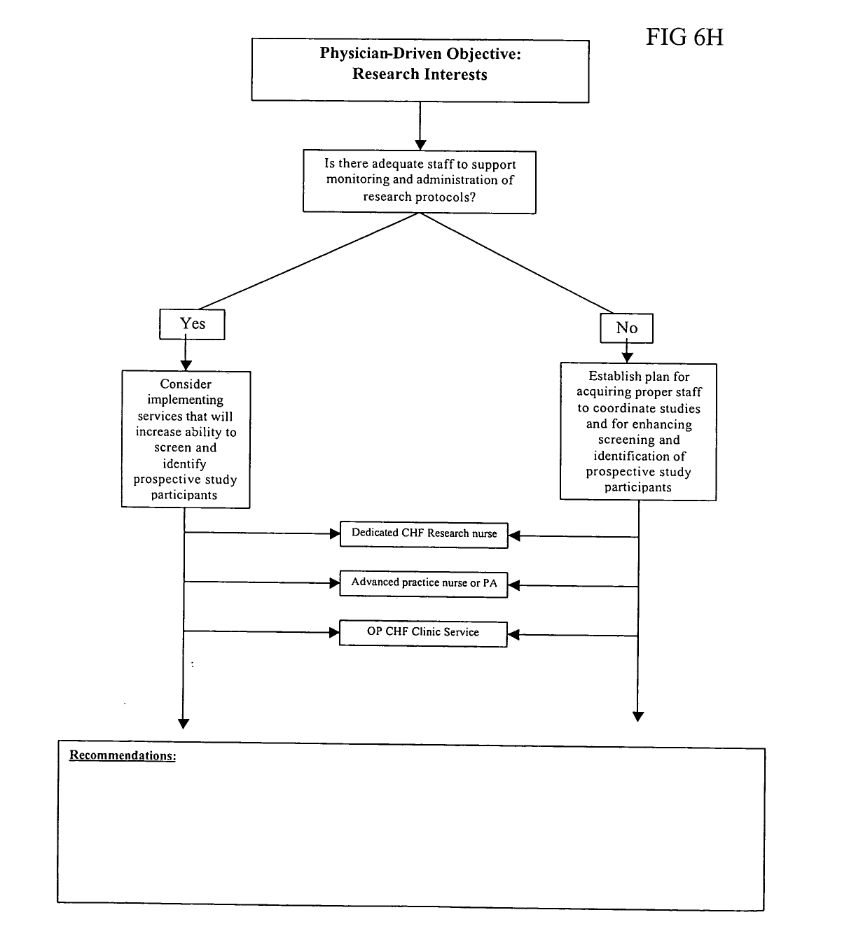

### DETAILED DESCRIPTION OF THE INVENTION

The present invention is a method and tool set to implement a disease management model for the operational, financial and clinical applications in the medical field. In this application the specialty of congestive heart failure is used as a detailed example. The method and tools provide the hospital with the directions needed to build an integrated heart failure program. Such a program bridges outpatient and inpatient care, tracks patients at home after discharge, uses an outpatient management protocol and tracking software, promotes continuous patient education, and accumulates data for payers and research projects. The present invention may be used to provide many benefits, some of which are listed below.

Bridges Outpatient and Inpatient Care. Most hospitals administer adequate inpatient care for heart failure patients. The acknowledged problem is what happens to the patient after discharge. In an integrated program, a heart failure clinic would seamlessly assume care of the patient and follow the patient as an outpatient.

Tracking Patients at Home. A special tracking software program may be utilized by heart failure clinic personnel to follow-up with the patient at home. A reporting schedule is established where the patient is expected to routinely contact the clinic with their vital signs and other pertinent data. In response to the patient input, protocol-directed management methods accumulated by the present invention and adopted, modified or developed by the program medical director(s), allow nurse practitioners (NP) to alter medications or dosages, diet and exercise. Patients that do not check-in at the agreed upon time will be flagged and contacted by the clinic. In the event the patient does not respond to treatment prescribed by the NP, arrangements will be made for them to enter the clinic for evaluation rather than directly admitted to the hospital or admitted through the emergency department (ED).

Continuous Patient Education. Patient education begins while the patient is hospitalized for heart failure. A clinic representative works with the hospital intensivist and floor nurses to ensure that the patient is educated about coping with their disease. The patient is seen by the clinic representative during hospitalization and at discharge. An interactive heart failure education manual is sent home with the patient. While at home, the patient is routinely contacted by a clinic representative.

Data Compilation and Analysis. Data is in compliance with HIPAA guidelines is obtained from the patient. The data accumulated on heart failure patients is subsequently used to assess the effectiveness of care, medical and surgical intervention, and outcomes for research investigation.

Congestive Heart Failure Program. The system provides an in-depth architecture for developing and implementing an integrated program in the hospital or clinic setting. Emphasis is on the analysis of the hospital's organizational support systems, current path of care for heart failure patients, reimbursement for heart failure visits and related procedures, and ancillary services.

The present invention builds from a database of diagnosis-based guidelines developed for medical professionals that can be used during various steps of the clinical decision process. (1) prospectively, before treatment, when an individual presents a health concern at the emergency room, doctors' office, clinic or operating room; (2) concurrently, at any stage of existing treatment; and (3) retrospectively, after treatment has been provided. The diagnosis-based guidelines are structured to work with an interactive question and answer methodology that ensures that the most appropriate data is collected and guides the user through the complex medical evaluation process. This is done by presenting questions in a logic-structured order, leading to guideline treatment option(s). The information retained by the system allows for a consistent, efficient review process. Variances between actual and proposed and guideline treatment option(s) can be used for quality assurance and audit purposes.

The present invention includes a processing unit and software-implemented health condition diagnosis-based guidelines. A user inputs an individual's health data into a new or existing case file in response to inquiries implemented in a health-condition specific guideline. Through the interactive guideline query-response process, one or more guideline treatment options are obtained. The user may adopt or accept the guideline treatment option(s) or input an actual or proposed treatment that is different. Discrepancies between the actual/proposed and guideline treatment option(s) are identified and the user's choice is documented through interactive queries. Once a treatment is selected, the case information is added to the database and a reviewer can analyze the file. The case may be re-opened, and changes may be made at any stage in the process to reflect new conditions or new or modified treatments.

In one example of the present invention, a method to improve quality of service at a heart failure clinic or unit at a hospital is performed. The method creates a recommendations and implementation guide by performing an assessment method designed to analyze the facility's resources and create a customized implementation strategy with the most effective clinic model and protocols. The heart failure recommendations and implementation guide addresses many factors. The factors may include some or all of the following menu of services:
Provider Outreach Function
Patient Search Function
Inpatient QI processes
Inpatient Heart Failure Unit
Hospital-based outpatient Heart Failure Clinic
MD practiced-based outpatient Heart Failure Clinic
Subacute Care Unit
24 hour OBS Unit
ED processes. communication from outpatient heart failure, pathways, OBS
Telemanagement
Case Management
Cardiac Rehab Services
QI tools, inpatient pathways, outpatient pathways, chart stamps, posters, staff and patient education materials, documentation pocket guides for indicators
Procedural follow-up. Surgical and EP

The present invention may also include patient tracking software. The present invention can use evidence based medicine in the form of the invention's patient tracking database, and the inventions clinical decision protocols and methods to effectively refer patients for the best treatment that as proven by the invention disease management systems. (e.g. A CHF patient and their doctor will now know the optimal medical therapy as of the date of diagnosis and if the patient is a good candidate for a pace maker, LVR procedure or other therapy.)

The patient tracking software could be used to track a patient's progress over time and throughout the therapies and procedures completed. The software is able to standardize the care of congestive heart failure patients by establishing immediate accessibility to criteria for disease classification and proposed therapy. The database utilizes all fields in the classification process. With a single query, a patient can be assigned a management strategy based on best practice models. The patient information, diagnosis and recommendations may be reviewed by the doctor and/or the patient throughout the therapeutic process. Further the patient symptom, diagnosis, prognosis, therapy selection, and outcomes could be used to further advance the accuracy of the tools of the present invention.

The program may be accessed through an automated service provider that is HIPAA compliant. Registered users can log on using any computer at the point of treatment or remotely as long as their system is equipped with an Internet browser. Users are given a password that dictates their level of access.

The present invention may also include marketing tools. The marketing tools may be used by the facility to promote their services to current and prospective patients. This may take many forms, including but not limited to written material or mailings, email campaigns, Internet advertising, and/or advertising space on a web interface of the present invention. In one embodiment, the marketing approach is a 3-pronged campaign designed to target family practitioners, cardiologists, surgeons, and heart failure patients.

Family practitioners (FPs) are the primary care providers for the patient's disease treatment pathway. Regional information campaigns are implemented consisting of e-mail outreach, direct mail, and lectures series on heart failure. The latest guidelines on care of the heart failure patient based on recommendations from the American College of Cardiology are included, as well as justification for enrolling patients into the hospital's heart failure program. Patients depend on the opinion of their family doctors. An informed family physician will be more inclined to send his or her patients to an integrated heart failure program.

Cardiologists have patients under their care that would benefit from participating in an integrated heart failure program. Similar outreach programs that are available for the family practitioner are also available for the cardiologist. Cardiologists will be more likely to admit their patients into the heart failure program if they are aware of the advantages.

The present invention may also be used by the family practitioners, cardiologists and other medical professionals to assist in educating patients about their condition, possible treatments, etc. The medical profession can refer the patient to particular sections of interest to the patient's condition, allowing the patient to later read more information and refer back the information if questions later arise.

Congestive heart failure patients may be given advertising material as a part of a direct-to-patient campaign that may be made available to hospitals or clinics as part of the marketing tool kit. Once the family practitioner and cardiologist are appropriately informed, the hospital will begin the advertising campaign that may include omni-media materials including but not limited to television, radio, and newsprint to create patient awareness and urge patients to the heart failure center.

Patients may also perform websearching and be directed to a website providing access to the diagnostic information in the database and/or give referrals to practitioners and specialists based on criteria selected by the patient. The criteria could include but are not limited to symptoms, procedures, success rates, proximity, etc. For example, a patient who knows or is interested in more information of their particular heart condition could search for a doctor that does procedures, treatments or therapies relating to LVR, TLVR (Thoracoscopic LVR), XLVR (Xiphoid LVR), PLVR, EndoAFib and chose the doctor based on patient selected criteria.

The patient may also obtain medical information from the interface. The information may take the form of a list of questions that can be used to query their treating physician as to treatment options and further information regarding procedures and specifics of their situation. Alternately, or in addition, upon entry of data into a query field, the patient is provided the latest peer reviewed articles on the related topic(s) and optionally a lay persons summary for the patient to provide to the treating physician. This would enable the patient to discuss why the patient believes a particular treatment is appropriate for their condition.

Several system and care modes are show in 5A-H. These are examples for integrated heart failure. The system and care models have been designed to accommodate the unique needs of individual hospitals as they build an integrated heart failure center. Once the site assessment tools have been completed, the method will identify the appropriate initial system model. Then, the model will be customized as needed. The model would then be given to the facility as a recommendation of the appropriate heart failure program model to implement.

Some possible embodiments and variations of the invention are listed below.

A system and method that connects a patient to their doctor, and the hospital wherein the patient, the doctor and the hospital are linked together and data gathered or provided by each is available for sharing and transmitting among the patient, his doctor and the hospital that is treating the patient. The system includes. a patient input means for entering disease health status data; a doctor input means for entering pre-selected billing and diagnosis data; a hospital input describing admission, discharge and billing information; and a decision making database engine that selects patients for a particular treatment based on previous billing data matching patients to prescribed treatments.

A method of treating a CHF patient wherein the best choice for the patient is selected from a surgical procedure, a cardiology procedure, or both procedures based on the condition of the patient, currently available treatment guidelines for the patient's condition and the best use of the resources, including the experience of the surgeon and the hospital in treating the patient using available and accepted treatment choices and determine the timing and order of each procedure.

A method for determining treatment for a CABG patient or CHF patient, wherein the patient is assessed for disease criteria as the basis for a decision to treat the patient with a cardiac surgery or cardiology procedure. The patient is then grouped into sub-treatment groups to determine the precise surgery of cardiology intervention procedure by a treatment algorithm based on accepted current medical guidelines, the treatment outcome is entered into the database to determine the best practice in the future and the treatment decision making database engine is automatically updated based on a pre-determined statistical power. Additionally, the method could be used by the hospital, where the hospital uses these methods to select a preferred medical device based on specific outcomes data for a specific treatment group.

An apparatus and method for determining treatment for a medical condition including a search means for enabling a patient to search for a health care provider, where the patient enters criteria into the search means, and the search provides the patient with the best match for their criteria.

The apparatus and method for determining treatment for a medical condition including a search means where patients are queried, and the search means searches for healthcare providers and doctors who can deliver the patient specified treatments.

The apparatus and method for determining treatment for a medical condition including a search means where a patient searches for a doctor via the Internet where patients and doctors are connected based on treatments offered and treatments desired.

The apparatus and method for determining treatment for a medical condition including a search means where patients can search for a doctor based on criteria that include types of procedures offered and experience.

The apparatus and method for determining treatment for a medical condition including a search means where the patient searches for a doctor based on the doctors specific documented outcomes for a specific procedure.

The apparatus and method for determining treatment for a medical condition including a search means where the patient searches for a doctor based on specific criteria determined by the patient such as cure rates and invasiveness of a procedure, or procedures.

The apparatus and method for determining treatment for a medical condition including a search means where the patient searched for a healthcare provider and a treatment based on peer review published data.

A method for patient self-diagnosis where the patient uses a remote automated query to accurately determine the disease state for their disease.

A method for patient self-diagnosis where a patient classifies themselves and then may search for treatment options based on disease status.

The method for patient self-diagnosis where the patient may then select treatments based on selected criteria such as cost, invasiveness, cure rates, and other criteria set by the patient. The selected criteria may include but is not limited to Medicare coverage, drug costs vs. surgery etc.

The method for patient self-diagnosis where patients may then select health care providers based on products and services offered by a provider, such as the Blue Egg, a St. Jude heart valve or pacemaker or a type of artificial hip, etc.

A medical education method for a patient to educate physicians where peer reviewed articles are loaded into a data base, abstracted into lay terms and made available to patients to print and present to their physician with both lay abstract and scientific abstracts available.

A medical education method where a patient, or healthcare provider may construct a treatment plan and present the plan to a doctor based on peer reviewed data entered into a searchable database where a treatment plan is located.

A clinical decision making system that uses a specific prospective clinical trial to determine the treatment strategy of a cardiovascular disease patient. eg. minimally invasive versus open heart surgery, beating heart versus stopped heart, beating heart supported versus fully arrested supported, coronary artery graft versus vein graft, stent versus drug eluting stent, minimally invasive LVR versus open LVR, percutaneous LVR versus minimally invasive LVR.

A clinical decision making system that provides a health care provider or physician the ability to use national, regional, or single hospital “best practices” and outcomes to determine the treatment and protocols for a given hospital or physician. This feature would data of interest to examine difference in regional practice. For example, a user could compare US practice to European practice, urban practice versus rural practice or HMO versus private payment versus Medicare.

The use of this disease management system by an insurance company or medicare to select best treatment alternatives for patient based on age, sex, or other predetermined criteria that are predetermined in the data base and clinical trials.

A portable wireless data collection terminal that allows patient and clinical personal to communicate critical patient information. This allows regular communication after the patient is released from the facility. For example, the system could be set up to automatically or with prompting question the patient about various things, such as but not limited to weight—Did you weigh? How are you on a scale of 1-10? What is your BNP level? Did you walk today?

A method of managing coronary artery disease for a patient including the steps of: (a) screening of a patient to determine the patient's risk for coronary artery disease; (b) providing education and preventative care for the patient based on the patient risk determined in step (a); (c) providing treatment for the patient, when the patient has symptomatic coronary artery disease; (d) and providing follow-up for the patient based on step (a) and step (c).

A method of managing coronary artery disease further including educational programs and/or outcome assessment.

A method of managing coronary artery disease wherein the treatment may include one or more of the following a diagnostic evaluation; least invasive coronary artery bypass surgery; interventional cardiology treatment, such as but not limited to percutaneous transluminal coronary angioplasty atherectomy and stent; alternative treatment; laser myoplasty; and surgical treatment.

A method of managing coronary artery disease wherein a plurality of least invasive coronary artery bypass surgical procedures are considered, and wherein the plurality of surgeries includes: (a) surgery performed in a closed chest environment on a beating heart with no cardiopulmonary support; (b) surgery performed in a closed chest environment on a beating heart with cardiopulmonary support; (c) surgery performed in a closed chest environment on a fibrillating heart with cardiopulmonary support; (d) surgery performed in a closed chest environment on an arrested heart with cardiopulmonary support; (e) and surgery performed in a open chest environment on an arrested heart with cardiopulmonary support.

A method of managing coronary artery disease wherein the treatment includes at least one of (a) least invasive coronary artery bypass surgery performed in a closed chest environment on a beating heart with no cardiopulmonary support; (b) least invasive coronary artery bypass surgery performed in a closed chest environment on a beating heart with cardiopulmonary support; (c) least invasive coronary artery bypass surgery performed in a closed chest environment on a fibrillating heart with cardiopulmonary support; (d) least invasive coronary artery bypass surgery performed in a closed chest environment on an arrested heart with cardiopulmonary support; and (e) least invasive coronary artery bypass surgery performed in a open chest environment on an arrested heart with cardiopulmonary support.

A method for selecting a treatment option for a patient with symptomatic coronary artery disease, including the steps of: (a) obtaining diagnostic cardiovascular information using a catheter introduced to the patient; and (b) and selecting a treatment option based on the diagnostic cardiovascular information, wherein the treatment option includes at least one of percutaneous treatment and least invasive coronary artery bypass surgery.

The method of determining treatment for a patient where a hospital uses one or more of the methods herein to select a preferred medical device based on specific outcomes data for a specific treatment group.

The method of determining treatment for a patient where a health care provider selects a treatment course based on the lowest cost treatment yielding a predicted outcome of life years.

The method of determining treatment for a patient where the doctor and patient are provided a list of health care providers in the data base that have the best outcome for the procedure selected by the method of determining treatment.

A method of determining treatment for a CHF patient that determines if a patient will have medical therapy alone, or a surgical procedure or a cardiology procedure, or both procedures as the best method of treatment for CHF, based on statistical significance.

A method for using outcome data for a specific treatment group and determining the interventions for a cardiac surgery whereby decisions such as whether the patient will have their mitral valve repaired or replaced, their left arterial appendage (LAA) removed, their cardiac tissue ablated to treat atrial fibrillation and if their coronary arteries will by surgically bypassed or stent(s) placed in their coronary arteries, are made.

A treatment method that selects a patient treatment by stage of disease or coexisting circumstances that preclude specific approaches. For example, percutaneous approach for Class II, Mini LVR for Class III, and LVR using Stemotomy for Class IV patients.

A method of determining treatment where the best treatment method is determined by the providers own outcomes data and a pre-selected publication data base, that is weighted by the provider, for a hybrid of best practices determined by the providers own outcomes and the outcomes of selected studies included in a database.

A patient tracking database and system that enables integrated care between the primary care doctor, a clinical specialist (in heart failure), a cardiologist, a surgeon, and the payer (Medicare or insurance company or HMO).

A clinical decision making protocol that determines the referral of a patient needing treatment for a cardiac disease to a cardiologist or a cardiac surgeon or another health care professional.

A clinical decision making software and database engine and protocol that enables a health care provider to determine the most effective initial treatment and sequence of patient referrals from a treating physician (cardiologist to surgeon/ER personnel to cardiologist to surgeon to cardiologist to heart failure clinical specialist) using evidence based medicine from a patient tracking database that is continuously updated with current best practices.

A disease management health status report for patients providing possible treatment options based on diagnostic test results provided to the patient.

A disease management template for patients to use in managing the treatment they receive from their treating physician, including options such as a list of questions that patients use to query their treating physicians as to treatment options, simplified explanations of conditions and treatments.

A clinical decision making system that personalizes the choice of treatment for the patient based on the patient's condition and choosing from commonly available options such as minimally invasive or open surgery; beating heart or stopped heart surgery; beating heart supported or fully arrested and supported; coronary artery graft, vein graft, bare metal stent or drug eluting stent; minimally invasive LVR or open LVR; percutaneous LVR or minimally invasive LVR procedure; and provides a health care provider or physician the ability to use national, regional, or single hospital “best practices” and outcomes to determine the treatment methods and protocols for a given hospital or physician.

The use of the disease management system by an insurance company or Medicare to select best treatment alternatives for patient, based on age, sex, or other established criteria that are predetermined in the database and clinical trials.

The use of the disease management system by an insurance company or Medicare to authorize payment for selected treatment alternatives based on the decision making system and database.

A portable wireless data collection terminal that will transparently inform a clinical specialist of their patient's health status, where the specialist will obtain the data (e.g., weight) from a wireless device (e.g., a wireless scale) with no active data entry by the patient and determine the patient's health status.

A data base patient tracking system the enables integrated care between the primary care doctor, a clinical specialist (in heart failure), a cardiologist, a surgeon, and the payer (medicare or insurance company or HMO).

A clinical decision making protocol that determines the referral of patient for a cardiac disease to cardiologist or cardiac surgeon or other health care professional.

A clinical decision making system and method that enables a health care provider to determine the most effective initial treatment and sequence of patient referrals using evidence based medicine from a patient tracking data base that is continuously updated with current best practices. This includes moves such as from cardiologist to surgeon, from ER personnel to cardiologist or to surgeon or to cardiologist or to heart failure clinical specialist.

A disease management health status report for patients providing possible treatment options based on diagnostic test results provided to the patient.

A clinical decision making system that uses a specific prospective clinical trial to determine the treatment strategy of a cardiovascular disease patient.

A clinical decision making system that provides a health care provider or physician the ability to use national, regional, or single hospital “best practices” and outcomes to determine the treatment and protocols for a given hospital or physician.

A portable wireless data collection terminal that will transparently inform a clinical specialist of their patients health status by using GPS the specialist will gain weight from a wireless scales with no input from the patient and determine if they are mobile to determine their health status.

A diagnostic questionnaire that uses a query analysis method for determining the accuracy of the patient responses to insure the accurate classification of CHF patients.

A diagnostic questionnaire where the classification of the stage of the CHF patient is verified by an MV02 test and the query analysis method is updated to optimize accuracy.

An integrated disease management process that uses best practices in the areas of drug therapy, surgery, and cardiology for an optimized total health plan for a disease.

The integrated disease management process where factors such as cost, or least invasive, or best long term outcome are used to determine the “optimized” treatment.

The integrated disease management process where patient inputs such as, surgeon experience, least invasive, insurance coverage or other critical patient criteria are used to determine the “optimal treatment”.

The integrated disease management process where insurance coverage alone determines the optimal mix of integrated care. (ie. If surgery is covered by Medicare and drugs are not the patient is aware of the option)

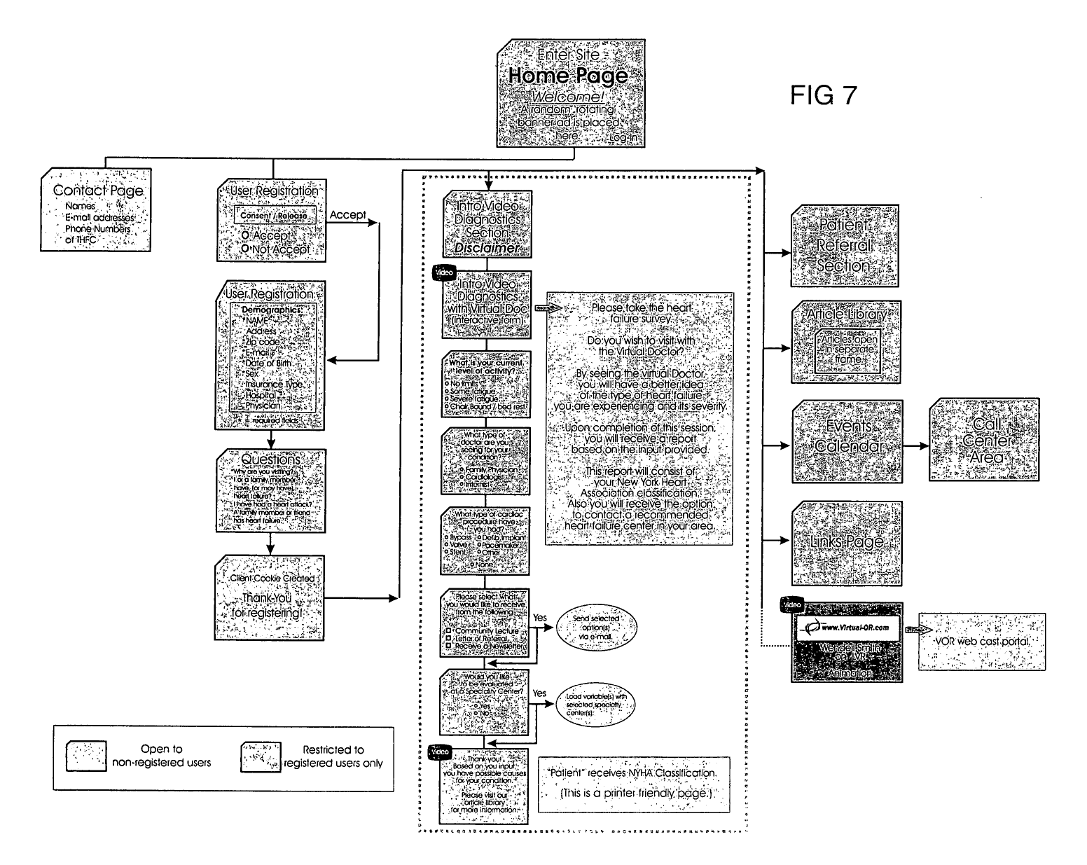

FIG. 7 is a flow chart of a web-based, interactive diagnosis, referral and reference system. In this embodiment, a patient would log onto the website of the present invention. In this embodiment of the website, a user would enter some preliminary information to register themselves as a user. Once registered, the user would have access to the diagnostic, referral and reference materials. If the patient had already been diagnosed, the patient could go directly to reference materials to review information on the condition. Alternately, the user could enter the name of the condition and review a list of suitable physicians to treat their condition. If undiagnosed or if additional confirmation of a diagnosis was desired, the user could answer a series of questions that would lead to a diagnosis based on the information entered by the user. The questions may take the form of a straight questionnaire with a preset group of questions. Alternately, a guided or intelligence series of questions that used the answer to prior questions to narrow down the topic and diagnosis could also be used. Once diagnosed, the user could then access either the reference and article library or the physician referral section.

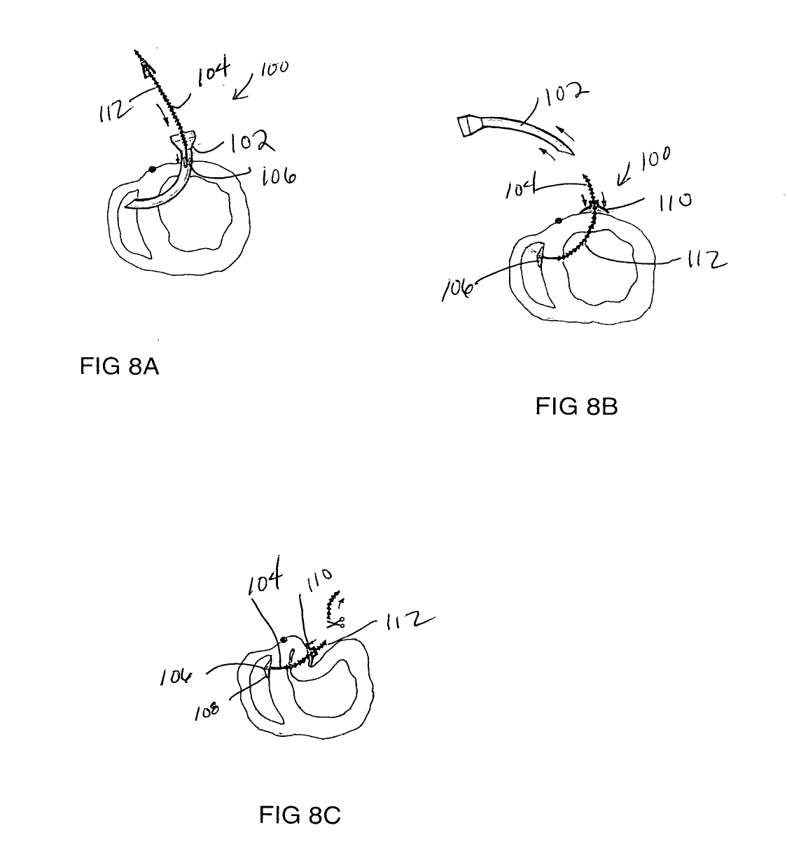

FIGS. 8A-C show a method and device 100 for left ventricular reconstruction using a left ventricular approach. The device 100 is guided to the correct location on the heart by introducing a hollow curved introducer 102 or needle in through the anterior wall of the left ventricle. A visual guidance system, such as TEE, may be used to ensure the placement accuracy. The tip of the curved needle 102 is then guided through the septum and into the right ventricle. Once the needle 102 is in place, the transventricular anchor 104 is loaded into the needle 102. The distal tip of the anchor 100 is extended into the right ventricle.

The distal tip of the anchor has a mechanical stop 106. Although it may take any suitable form, in the embodiment shown, the stop 106 has two or more arms 108. While the anchor 100 is within the needle 102, the arms 108 are held close to the body of the anchor 100. When the distal tip of the anchor 100 extends beyond the distal tip of the needle 102, the arms 108 are free to open. Once opened, the arms 108 prevent the distal end of the anchor 100 from passing back through the opening in the septum.

After the anchor 100 is in place, the needle 102 may be removed. A proximal sealing lock 110 is then slid onto the proximal end of the anchor 100. The sealing lock 110 is slid along the body and over one or more barbs or other protrusions 112 extending from the body of the anchor 100. The barbs 112 may take any suitable form, such as rounded or triangular. In the embodiment shown, the barbs 112 are generally triangular in shape. The proximal sealing lock 110 is advance until the anterior wall of the left ventricle is pressed inward, thereby folding the wall and reducing the interior volume of the left ventricle. Once the sealing lock 110 is advanced into place, the proximal portion of the body of the anchor 100 may be cut off.

If desired, the hollow needle tip 102 may be equipped with a pressure sensor to guide the practitioner to know if the device is in the left ventricle, septum or the right ventricle by sensing the pressure. The hollow needle 102 may also be equipped with electrical sensor (EKG, Monophasic Action Potential) to sense if the puncture sight is the viable tissue or infarcted tissue.

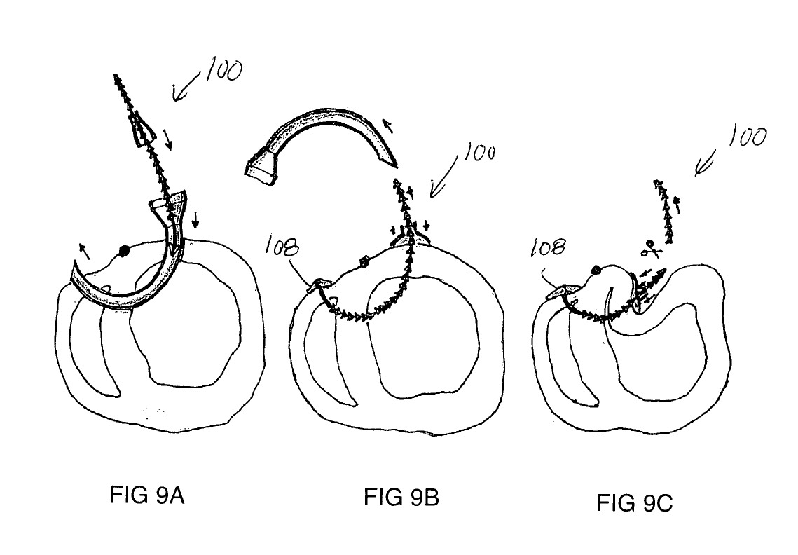

FIGS. 9A-C show an alternate procedure of that shown in FIGS. 8A-C. In this method, the distal end and mechanical stop 108 of the anchor 100 is passed from the right ventricle back outside the anterior wall of the heart.

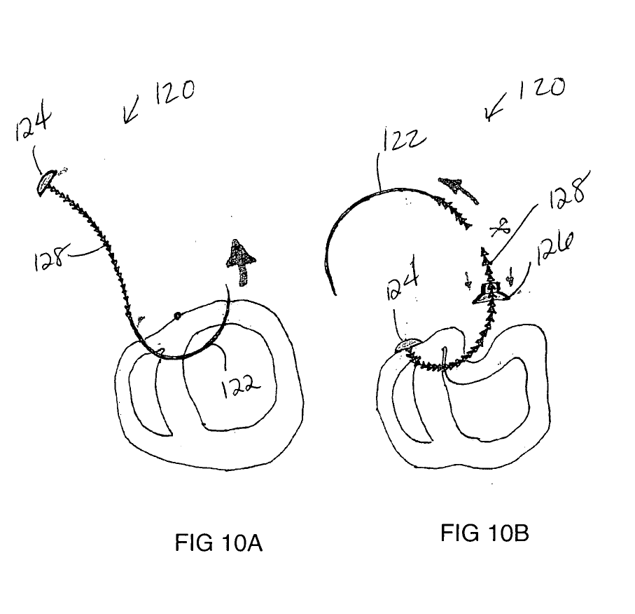

FIGS. 10A-B show a method and device for left ventricular reconstruction using a right ventricular approach. In this version, a curved needle 122 forms the distal tip of the anchor 120. The curved needle 122 is inserted through the anterior wall and into the right ventricle, through the septum, and through the anterior wall of the left ventricle. The body of the anchor 120 follows the curved needle 122 and is feed through until the proximal stop 124 engages the anterior surface of the right ventricle. The anterior wall of the left ventricle is pressed inward over the body of the anchor. A distal sealing stop 126 is threaded over the anchor 120 and slid in place against the anterior surface of the right ventricle. The heart tissue may be pressed inward to fold the wall of the heart prior to the placement of the sealing stop 126 or the sealing stop 126 may be used to manipulate the heart tissue. A plurality of barbs 128 or protrusions extends from the surface of the anchor body. The barbs 128 help hold the heart tissue in place. The curved needle 122 and the excess portion of the distal end of the anchor 120 may be removed. This may be done before or after the distal sealing stop 126 has been placed.

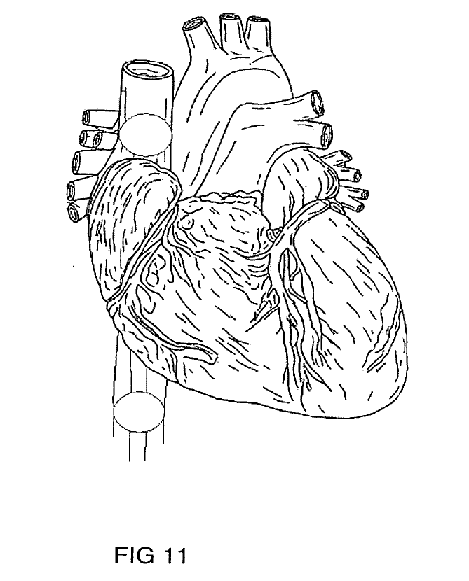

FIG. 11 shows unloading of the left ventricle. The left ventricle is unload by closing off the blood supply to the lungs via right ventricle, therefore, less blood supply will be going to the left ventricle, thereby unloading the left ventricle.

Many features have been listed with particular configurations, options, and embodiments. Any one or more of the features described may be added to or combined with any of the other embodiments or other standard devices to create alternate combinations and embodiments.

Although the invention has been fully described above, in relation to various exemplary embodiments, various additions or other changes may be made to the described embodiments without departing from the scope of the present invention. Thus, the foregoing description has been provided for exemplary purposes only and should not be interpreted to limit the scope of the invention.

What is claimed is:

1. A method of diagnosis and patient-initiated referral for treating a patient, the method comprising the steps of:
(a) a patient accessing a webpage;
(b) the patient reviewing information on the webpage;
(c) and the patient using the webpage to obtain a referral to one of a plurality of doctors listed on the webpage.

2. The method of claim 1, further comprising the steps of:
(d) the patient answering a series of questions regarding a condition;
(e) the webpage displaying one or more preliminary diagnoses;
and wherein the plurality of doctors listed are selected based on the one or more preliminary diagnoses displayed in step (e).

3. The method of claim 1, used to diagnose and self-refer a patient having heart related conditions.

4. The method of claim 1, used to diagnose and self-refer a patient having coronary artery disease.

5. The method of claim 1, used to diagnose and self-refer a patient needing heart reconstruction.

6. The method of claim 5, wherein a ventricular reconstruction procedure is performed, the procedure comprising the steps of:
(d) passing a curved needle through an anterior wall of a left ventricle of a patient;
(e) passing the curved needle through the septum and into the right ventricle;
(f) inserting an anchor into the needle;
(g) allowing one or more arms located on a distal end of said anchor to extend;
(h) removing the curved needle;
(i) placing a sealing member over a proximal end of said anchor;
(j) folding a wall of the ventricle inward;
(k) using the sealing member to hold the folded wall in place.

7. The method of claim 6, wherein said curved needle is also passed through an anterior wall of the right ventricle prior to step (g).
